(19) United States
(12) Patent Application Publication
(10) Pub. No.: US 2024/0425703 A1
(43) Pub. Date: Dec. 26, 2024

(54) Preparation and Application of Light-resistant Fluorane Chromotropic Dye

(71) Applicant: Jiangnan University, Wuxi (CN)

(72) Inventors: Liping ZHANG, Wuxi (CN); Weize WU, Wuxi (CN); Shaohai FU, Wuxi (CN); Yu GUAN, Wuxi (CN)

(21) Appl. No.: 18/814,350

(22) Filed: Aug. 23, 2024

Related U.S. Application Data
(63) Continuation of application No. PCT/CN2024/086554, filed on Apr. 8, 2024.

(30) Foreign Application Priority Data
Jan. 2, 2024 (CN) 2024100025021

(51) Int. Cl. C09B 11/24; B01J 13/20

(52) U.S. Cl. CPC C09B 11/24; B01J 13/206

(57) ABSTRACT

The present disclosure discloses preparation and application of a light-resistant fluorane chromotropic dye and belongs to the technical field of fine chemical engineering. By optimizing a structure of a commercial fluorane dye, the present disclosure provides a preparation method for a fluorane dye with a full spectrum and good light resistance. On the basis of maintaining a color rendering property, discoloration sensitivity, discoloration fatigue resistance and other properties of the dye, the light resistance is improved, a light resistance time is prolonged by 8-28 hours and is increased by 50% to 80%, a main problem of application of such temperature-sensitive materials in the field of functional textiles is solved, the application field is expanded, and economic benefits are facilitated.

### TECHNICAL FIELD

The present disclosure relates to preparation and application of a light-resistant fluorane chromotropic dye and belongs to the technical field of fine chemical engineering.

### BACKGROUND

After surfaces or interiors of materials are colored, chromotropic dyes can endow the materials with characteristics of discoloration with external factors, such as light, heat or other physical factors. According to structural features, the chromotropic dyes mainly have seven types, including phenolphthalein dyes, fluorane dyes, triarylmethane dyes, phenazines, thiazines, quinones, tetrazolium salts and spiropyrans.

The fluorane dyes, due to multiple response characteristics (such as temperature response, light response, humidity response, electroresponse, pH response and multi-stimulus response), have been widely used in smart textiles, decorations, sensors, displays and other fields. However, the fluorane dyes have poor light resistance, such that development and application of the fluorane dyes in long-term outdoor products are limited. Therefore, development of light-resistant fluorane dye chromotropic systems has always been a focus of research.

At present, ultraviolet absorbers, due to an ability to absorb ultraviolet light, have been introduced into thermochromic microcapsule shells or smart textile surfaces to achieve an effect of protecting dye structures from light damage. For example, an article Applied Surface Science 442 (2018): 71-77 reports that with 2-[3-(2H-benzotriazol-2-yl)-4-hydroxyphenyl]ethyl methacrylate (BHEM), vinyltrimethoxysilane (VTMS) and hexafluorobutyl methacrylate (HFMA) as modified monomers, a fluorosilicon acrylic resin polymer with an ultraviolet absorption property is successfully prepared by a solution polymerization method to improve the light resistance; and in a Chinese patent CN114808472A, a thermochromic microcapsule with a core-shell structure is prepared by an emulsion polymerization method, where a polymer on a shell layer of the microcapsule has an anti-ultraviolet property, such that a color fading rate of the thermochromic microcapsule is 60.9% lower than that of conventional chromotropic microcapsules. However, only simply encapsulation and coating protection of the fluorane dyes to improve the light resistance cannot achieve long term effectiveness. Therefore, in addition to improving the light resistance with external forces, many scholars have also tried to obtain the light resistance by designing and optimizing molecular structures. For example, in an article Dyes and Pigments 190 (2021): 109294, a fluorescent cationic coumarin dye with a rigid molecular structure is synthesized to improve the light resistance; and the article Dyes and Pigments 197 (2022): 109924 reports that a fluorane dye containing a long alkyl chain fluorescent group has good light resistance. Therefore, molecular structures of the fluorane chromotropic dyes need to be optimized and designed to obtain excellent light resistance.

### SUMMARY

### Technical Problems

Fluorane chromotropic dyes have poor light resistance, which lose color rendering and discoloration abilities after being exposed to sunlight for a week and thus cannot meet use requirements of people. For this reason, a fluorane chromotropic dye with light resistance and an excellent structure needs to be designed and synthesized.

### Technical Schemes

The present disclosure mainly designs a fluorane chromotropic dye containing alkylamino, and that is to say, on the basis of a parent structure of an existing fluorane dye, the alkylamino is introduced to change an agglomeration state of a molecular structure of the dye by way of an easy agglomeration property so as to improve the light resistance. Alkyl is an alkyl chain with different carbon atom numbers (C₂-C₁₂), a cycloalkane with different carbon atom numbers, or an alkyl hydrocarbon group containing some polar groups (for example, the polar groups include O, CO, NH, CO—NH, etc.).

A first purpose of the present disclosure is to provide a fluorane chromotropic dye with good light resistance. The fluorane chromotropic dye has a structure shown in a formula I below:

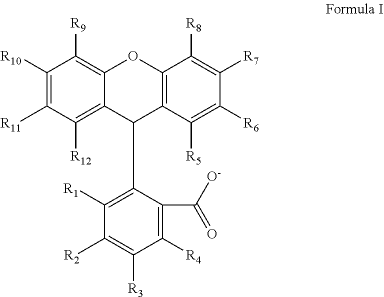

where:
R₅, R₆, R₈, R₉, R₁₁ and R₁₂ are each independently selected from hydrogen, alkyl, substituted alkyl, alkenyl, substituted alkenyl, heterocyclic alkyl, substituted heterocyclic alkyl, alkoxyl, substituted alkoxyl, substituted carbonyl, acylamino, substituted aminocyclohexane, and halogen;
R₇ is independently selected from N,N-diethyl, methoxyl, N,N-di(p-methylphenyl) yl, or chloro;
R₁₀ is independently selected from N,N-diethyl, methoxyl, N,N-di(p-methylphenyl) yl, or N-cyclohexyl; and
any 1-2 of R₁, R₂, R₃ and R₄ are alkylamino with different carbon atom numbers, where alkyl in the alkylamino is a straight-chain alkane with a carbon atom number of C₂-C₁₂, a cycloalkane, or an alkane containing a polar group (the polar group includes O (ether bond), CO (carbonyl), NH (imino), or CO—NH₂ (amido), etc.); and the remaining groups are selected from one or more of hydrogen, alkyl, substituted alkyl, alkenyl, substituted alkenyl, heterocyclic alkyl, and substituted heterocyclic alkyl.

In one embodiment of the present disclosure, the fluorane chromotropic dye has a general structural formula shown in a formula II below:

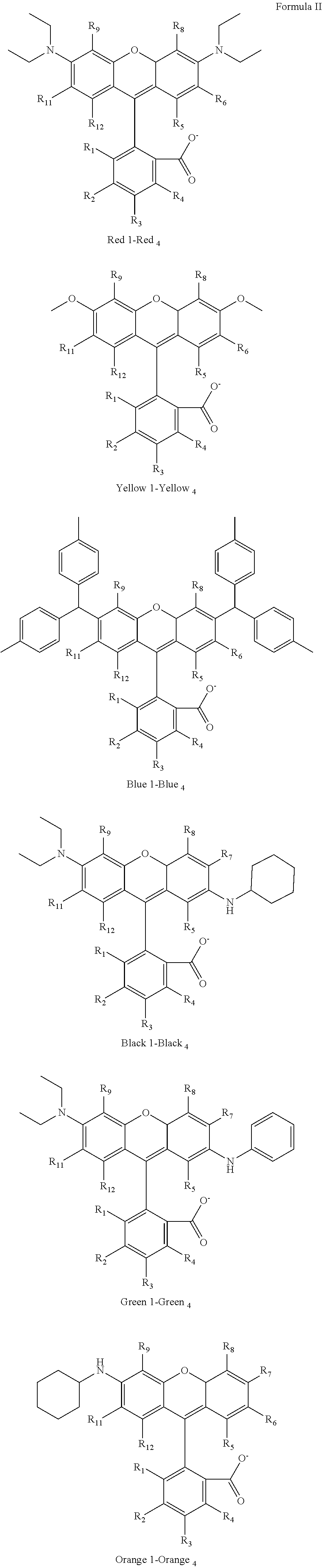

where:
R₅, R₆, R₇, R₈, R₉, R₁₁ and R₁₂ are each independently selected from hydrogen, alkyl, substituted alkyl, alkenyl, substituted alkenyl, heterocyclic alkyl, substituted heterocyclic alkyl, alkoxyl, substituted alkoxyl, substituted carbonyl, acylamino, and halogen;
R₁ and R₄ are H; and
R₂ and R₃ are independently selected from hydrogen or alkylamino with different carbon atom numbers, where alkyl in the alkylamino is a straight-chain alkane with a carbon atom number of C₂-C₁₂, a cycloalkane, or an alkane (C₂-C₁₂) containing a polar group; and the R₂ and the R₃ are not hydrogen simultaneously.

Preferably, the R₂ and the R₃ are independently selected from hydrogen or alkylamino, where alkyl in the alkylamino includes ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, and alkyl (C₂-C₁₀) containing a polar group; and the R₂ and the R₃ are not hydrogen simultaneously.

Further preferably, the R₂ and the R₃ are independently selected from hydrogen or alkylamino, where alkyl in the alkylamino includes ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, and alkyl (C₂-C₁₀) containing any one of hydroxyl or carboxyl; and the R₂ and the R₃ are not hydrogen simultaneously.

The fluorane chromotropic dye with good light resistance provided by the present disclosure is applied in the fields of dyes, textiles, clothing, printing and painting.

A second purpose of the present disclosure is to provide a method for improving sunlight resistance of a chromotropic material. The method includes coloring a material with a fluorane chromotropic dye, and the fluorane chromotropic dye has a general structural formula shown in a formula III below:

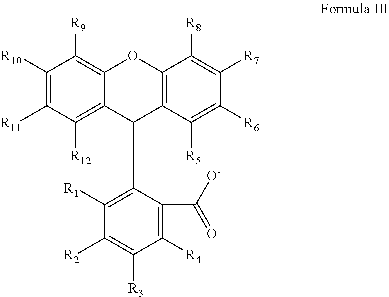

where:
R₅, R₆, R₈, R₉, R₁₁ and R₁₂ are each independently selected from hydrogen, alkyl, substituted alkyl, alkenyl, substituted alkenyl, heterocyclic alkyl, substituted heterocyclic alkyl, alkoxyl, substituted alkoxyl, substituted carbonyl, acylamino, substituted aminocyclohexane, and halogen;
R₇ is independently selected from N,N-diethyl, methoxyl, N,N-di(p-methylphenyl) yl, or chloro;
R₁₀ is independently selected from N,N-diethyl, methoxyl, N,N-di(p-methylphenyl) yl, or N-cyclohexyl; and
any 1-2 of R₁, R₂, R₃ and R₄ are alkylamino with different carbon atom numbers, where alkyl in the alkylamino is a straight-chain alkane with a carbon atom number of C₂-C₁₂, a cycloalkane, or an alkane containing a polar group (the polar group includes O (ether bond), CO (carbonyl), NH (imino), or CO—NH₂ (amido), etc.); and the remaining groups are selected from one or more of hydrogen, alkyl, substituted alkyl, alkenyl, substituted alkenyl, heterocyclic alkyl, and substituted heterocyclic alkyl.

In one embodiment of the present disclosure, the fluorane chromotropic dye has a general structural formula shown in a formula IV below:

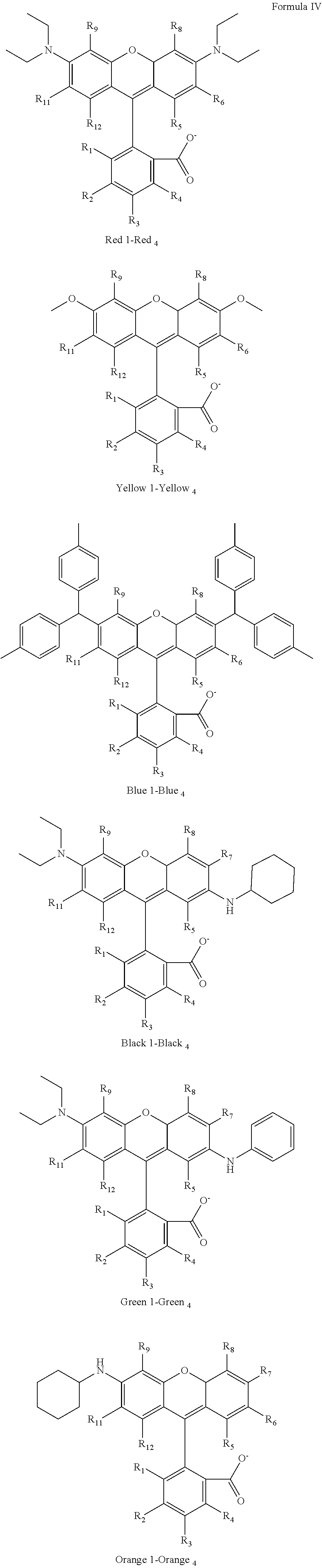

where:
R₅, R₆, R₇, R₈, R₉, R₁₁ and R₁₂ are each independently selected from hydrogen, alkyl, substituted alkyl, alkenyl, substituted alkenyl, heterocyclic alkyl, substituted heterocyclic alkyl, alkoxyl, substituted alkoxyl, substituted carbonyl, acylamino, and halogen;
R₁ and R₄ are H; and
R₂ and R₃ are independently selected from hydrogen or alkylamino with different carbon atom numbers, where alkyl in the alkylamino is a straight-chain alkane with a carbon atom number of C₂-C₁₂, a cycloalkane, or an alkane (C₂-C₁₂) containing a polar group; and the R₂ and the R₃ are not hydrogen simultaneously.

Preferably, the R₂ and the R₃ are independently selected from hydrogen or alkylamino, where alkyl in the alkylamino includes ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, and alkyl (C₂-C₁₀) containing a polar group; and the R₂ and the R₃ are not hydrogen simultaneously.

Further preferably, the R₂ and the R₃ are independently selected from hydrogen or alkylamino, where alkyl in the alkylamino includes ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, and alkyl (C₂-C₁₀) containing any one of hydroxyl or carboxyl; and the R₂ and the R₃ are not hydrogen simultaneously.

In one embodiment of the present disclosure, the material includes textile materials, composite materials, display materials, sensing materials, and painting materials, etc.

In one embodiment of the present disclosure, the textile materials refer to fibers and fiber products, including fibers, yarns, fabrics, non-woven fabrics and composites thereof.

A third purpose of the present disclosure is to provide a synthetic method for the fluorane chromotropic dye, which includes the following steps:
mixing a fluorane chromotropic dye structural intermediate with alkylamine, then adding an organic solvent and a catalyst, performing stirring, adjusting a pH value to 8-9, then carrying out a heating reaction, and after the reaction is completed, performing purification to obtain the fluorane chromotropic dye.

In one embodiment of the present disclosure, the synthetic method needs to be carried out in a protective gas environment.

In one embodiment of the present disclosure, the protective gas includes one or more of an inert gas or nitrogen.

In one embodiment of the present disclosure, the fluorane chromotropic dye structural intermediate has a general structural formula shown in a formula V below:

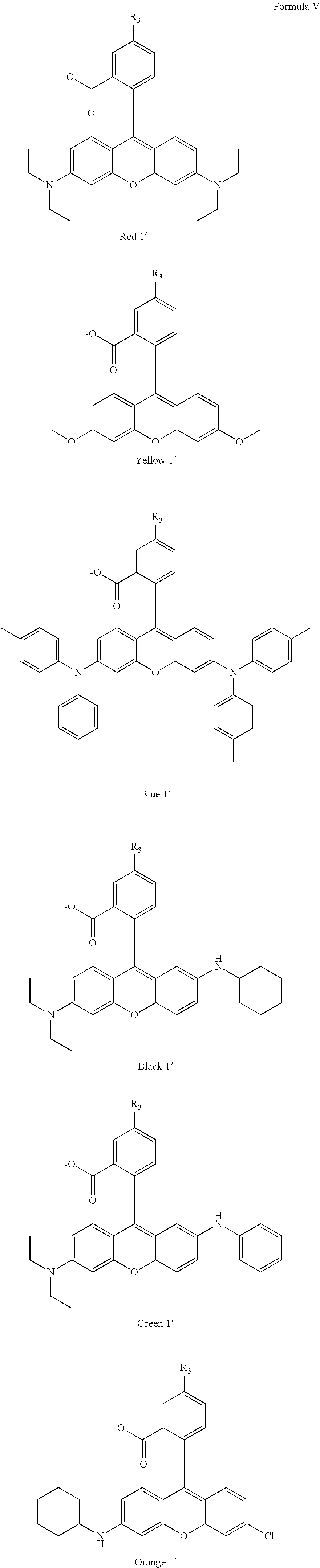

where: R₃ is halogen.

Further, the halogen is Cl, Br, or F.

In one embodiment of the present disclosure, the fluorane chromotropic dye structural intermediate may also have a general structural formula shown in a formula VI below:

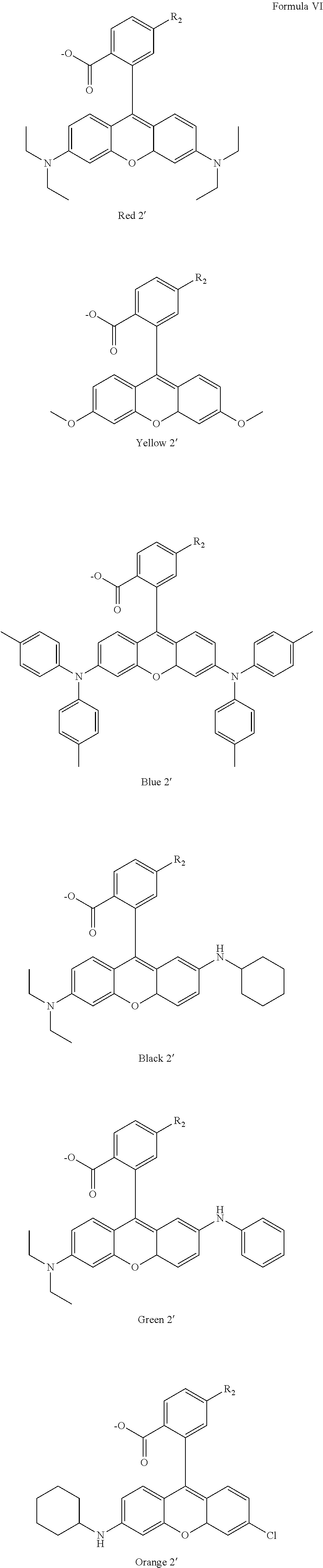

where: R₂ is halogen.

Further, the halogen is Cl, Br, or F.

In one embodiment of the present disclosure, alkyl in the alkylamine includes a C₂-C₁₂ straight-chain alkane, a cycloalkane, or an alkyl hydrocarbon group containing a polar group.

In one embodiment of the present disclosure, the polar group includes O (ether bond), CO (carbonyl), NH (imino), or CO—NH₂ (amido).

In one embodiment of the present disclosure, the organic solvent is toluene.

In one embodiment of the present disclosure, the catalyst is niobium pentoxide.

In one embodiment of the present disclosure, a molar ratio of the fluorane chromotropic dye structural intermediate to the alkylamine is 1:(1-2).

In one embodiment of the present disclosure, a ratio of a molar amount of the fluorane chromotropic dye structural intermediate to a volume of the organic solvent is 1 mol:(0.05-0.1) mL.

In one embodiment of the present disclosure, a molar ratio of the fluorane chromotropic dye structural intermediate to the catalyst is 1:(0.5-0.7).

In one embodiment of the present disclosure, the heating reaction is carried out at 75-85° C. for 6-9 h.

In one embodiment of the present disclosure, the purification may be performed by column chromatography or gas chromatography.

A fourth purpose of the present disclosure is to provide a sunlight-resistant chromotropic microcapsule. The sunlight-resistant chromotropic microcapsule adopts the fluorane chromotropic dye as a core material and a high-molecular polymer, an inorganic particle or an inorganic particle doped polymer as a wall material.

In one embodiment of the present disclosure, a preparation method for the chromotropic microcapsule includes the following steps:
(1) preparation of a prepolymer: taking urea and a formaldehyde solution for mixing, then adding triethanolamine to adjust the pH value to 8-9, and carrying out a heating reaction to obtain a viscous transparent urea-formaldehyde prepolymer;
(2) dispersion of a capsule core: mixing the fluorane chromotropic dye, bisphenol A and a solvent to prepare the capsule core, then mixing the capsule core with water, performing shearing dispersion emulsification, and performing continued dispersion after cooling to form an O/W type emulsion; and
(3) microencapsulation and post-treatment: mixing the viscous transparent urea-formaldehyde prepolymer with the O/W type emulsion to obtain a mixed solution, then adding sodium chloride and silica for full stirring, then adding acetic acid to adjust the pH value to 3-4, followed by carrying out a heating reaction, and after the reaction is completed, performing cooling, washing, filtering and drying to obtain the chromotropic microcapsule.

Further, the heating reaction in step (1) includes performing heating to 70-80° C. to carry out a reaction for 1-2 h.

Further, in step (2) of the preparation method, the solvent is a mixed solution of tetradecanol and hexadecanol that are mixed at a mass ratio of (4-5):(5-6).

Further, in step (2) of the preparation method, a mass ratio of the fluorane chromotropic dye to the bisphenol A is 1:(2-8).

Further, in step (2) of the preparation method, a mass ratio of the fluorane chromotropic dye to the solvent is 1:(50-70).

Further, in step (2) of the preparation method, a volume ratio of the capsule core to the water is 1:(4-7).

Further, in step (2) of the preparation method, a shearing speed is 1,000-3,000 r/min, and a time is 1-2 h.

Further, in step (2) of the preparation method, the shearing dispersion emulsification is performed at 60-70° C.; and the cooling is performed to 30-40° C.

Further, in step (2) of the preparation method, the continued dispersion is performed for 1-2 h.

Further, in step (3) of the preparation method, a volume ratio of the viscous transparent urea-formaldehyde prepolymer to the O/W type emulsion is (5-10): 1.

Further, in step (3) of the preparation method, the added sodium chloride accounts for 0.2-0.25 wt % of a total mass of the mixed solution.

Further, in step (3) of the preparation method, the added silica accounts for 0.2-0.25 wt % of the total mass of the mixed solution.

Further, in step (3) of the preparation method, the heating reaction is carried out at 60-65° C. for 30-50 min.

The chromotropic microcapsule provided by the present disclosure is applied in the fields of textiles and dyeing.

A fifth purpose of the present disclosure is to provide a preparation method for a sunlight-resistant chromotropic fabric. The method includes first preparing a thermochromic color paste by using the microcapsule prepared from the fluorane chromotropic dye, then coloring a fabric with the thermochromic color paste, and finally obtaining a chromotropic fabric with sunlight resistance.

In one embodiment of the present disclosure, the fabric includes fibers, cotton fabrics, silk fabrics, or polyester textiles.

In one embodiment of the present disclosure, composition of the thermochromic color paste includes a thickener, an adhesive, a chromotropic microcapsule, and water.

In one embodiment of the present disclosure, a mass ratio of the thickener to the adhesive is 1:(5-15).

In one embodiment of the present disclosure, a mass ratio of the thickener to the chromotropic microcapsule is 1:(60-100).

In one embodiment of the present disclosure, a mass ratio of the thickener to the water is 1:(5-15).

In one embodiment of the present disclosure, a preparation process of the sunlight-resistant chromotropic fabric includes:

fixing the fabric, then subjecting the fabric to printing treatment by using a screen printing technology, then drying the printed fabric at 70-90° C., and performing curing at 90-100° C. for 2-5 min to obtain the sunlight-resistant chromotropic fabric.

The present disclosure provides a chromotropic fabric with sunlight resistance that is prepared according to the method.

The chromotropic fabric with sunlight resistance provided by the present disclosure is applied in the fields of clothing and textiles.

The present disclosure has the following beneficial effects.

By optimizing a structure of a commercial fluorane dye, the present disclosure provides a preparation method for a fluorane dye with a full spectrum and good light resistance. On the basis of maintaining a color rendering property, discoloration sensitivity, discoloration fatigue resistance and other properties of the dye, the light resistance is improved, the light resistance time is prolonged by 8-28 hours and is increased by 50% to 80%, a main problem of application of such temperature-sensitive materials in the field of functional textiles is solved, the application field is expanded, and economic benefits are facilitated.

### DETAILED DESCRIPTION

Preferred embodiments of the present disclosure are described below. It should be understood that the embodiments are intended to better explain the present disclosure, rather than to limit the present disclosure.

### 1. Experimental Materials

Monomers used in the examples include 4-bromophthalic anhydride and m-hydroxy-N,N-diethylaniline, with CAS numbers of 86-90-8 and 91-68-9, respectively, which are purchased from Shanghai Macklin Biochemical Technology Co., Ltd.; and all other chemicals are commercially available products.

### 2. Test Methods

(1) Sunlight resistance test of a fluorane chromotropic dye: The fastness to sunlight of a self-made dye phase change material is tested according to an American AATCC TM16 light fastness test standard, discoloration conditions of the material are observed, an apparent depth (K/S value) of the material is measured by an UltraScanXE computer color measurement instrument, and then an average value is calculated. During the test, a D65 light source with an observation angle of 10° is used.

(2) Color fading rate: Sunlight aging of a chromotropic fabric is artificially simulated by using a sunlight climate testing machine, and calculation is performed according to the following formula in combination with the K/S value of the fabric at different sunlight exposure times:

$$Color fading rate = [ \frac{K / {S}_{0} - K / {S}_{t}}{K / {S}_{0}} ] \times 100 \%$$

In the formula: K/S₀ is an initial K/S value of the fabric, and K/St is a K/S value of the fabric after t hours.

### Example 1: A Fluorane Chromotropic Dye Structure with Good Light Resistance and a Synthetic Method Therefor

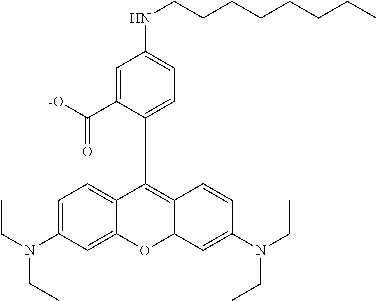

Under the protection of N₂, red 1′ (where R was —Cl) and octylamine were added to a 100 mL three-neck flask at a molar ratio of 1:1.5, then toluene was added to make the red 1′ and the octylamine have a concentration of 0.02 mol/L and 0.03 mol/L, respectively, and a niobium pentoxide catalyst that was 50% of a molar equivalent of the red 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, red 1.

### Example 2: A Fluorane Chromotropic Dye Structure with Good Light Resistance and a Synthetic Method Therefor

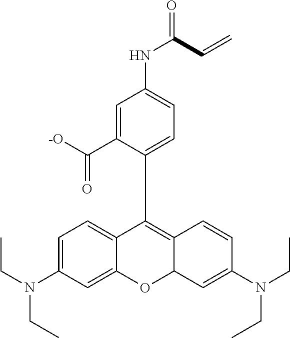

Under the protection of N₂, red 1′ (where R was —Cl) and acrylamide were added to a 100 mL three-neck flask at a molar ratio of 1:1.5, then toluene was added to make the red 1′ and the acrylamide have a concentration of 0.02 mol/L and 0.03 mol/L, respectively, and a niobium pentoxide catalyst that was 50% of a molar equivalent of the red 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, red 2.

### Example 3: A Fluorane Chromotropic Dye Structure with Good Light Resistance and a Synthetic Method Therefor

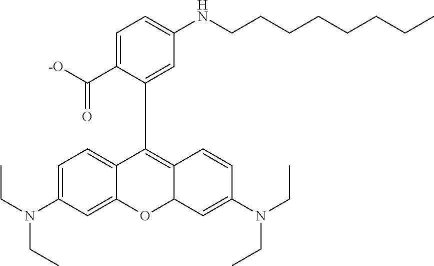

Under the protection of N₂, red 2′ (where R was —Cl) and octylamine were added to a 100 mL three-neck flask at a molar ratio of 1:1.5, then toluene was added to make the red 2′ and the octylamine have a concentration of 0.02 mol/L and 0.03 mol/L, respectively, and a niobium pentoxide catalyst that was 50% of a molar equivalent of the red 2′ and 50 ml of a toluene organic solvent were added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, red 3.

### Example 4: A Fluorane Chromotropic Dye Structure with Good Light Resistance and a Synthetic Method Therefor

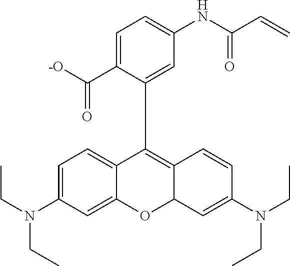

Under the protection of N₂, red 2′ (where R was —Cl) and acrylamide were added to a 100 mL three-neck flask at a molar ratio of 1:1.5, then toluene was added to make the red 2′ and the acrylamide have a concentration of 0.02 mol/L and 0.03 mol/L, respectively, and a niobium pentoxide catalyst that was 50% of a molar equivalent of the red 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, red 4.

### Example 5: A Fluorane Chromotropic Dye Structure with Good Light Resistance and a Synthetic Method Therefor

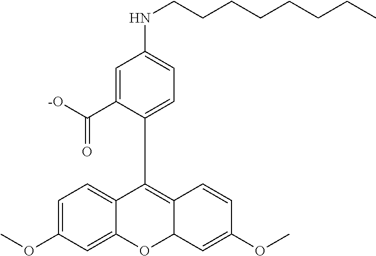

Under the protection of N₂, yellow 1′ (where R was —Cl) and octylamine were added to a 100 mL three-neck flask at a molar ratio of 1:1.5, then toluene was added to make the yellow 1′ and the octylamine have a concentration of 0.02 mol/L and 0.03 mol/L, respectively, and a niobium pentoxide catalyst that was 50% of a molar equivalent of the yellow 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, yellow 1.

### Example 6: A Fluorane Chromotropic Dye Structure with Good Light Resistance and a Synthetic Method Therefor

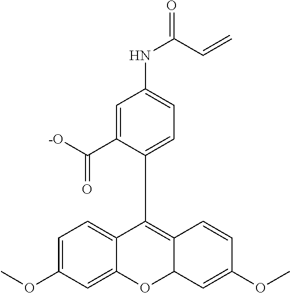

Under the protection of N₂, yellow 1′ (where R was —Cl) and acrylamide were added to a 100 mL three-neck flask at a molar ratio of 1:1.5, then toluene was added to make the yellow 1′ and the acrylamide have a concentration of 0.02 mol/L and 0.03 mol/L, respectively, and a niobium pentoxide catalyst that was 50% of a molar equivalent of the yellow 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, yellow 2.

### Example 7: A Fluorane Chromotropic Dye Structure with Good Light Resistance and a Synthetic Method Therefor

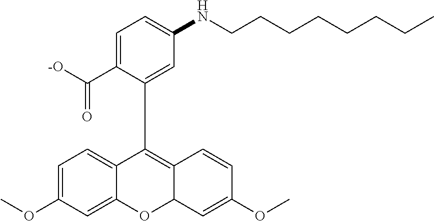

Under the protection of N₂, yellow 2′ (where R was —Cl) and octylamine were added to a 100 mL three-neck flask at a molar ratio of 1:1.5, then toluene was added to make the yellow 2′ and the octylamine have a concentration of 0.02 mol/L and 0.03 mol/L, respectively, and a niobium pentoxide catalyst that was 50% of a molar equivalent of the yellow 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, yellow 3.

### Example 8: A Fluorane Chromotropic Dye Structure with Good Light Resistance and a Synthetic Method Therefor

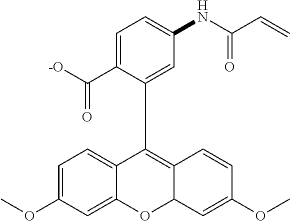

Under the protection of N₂, yellow 2′ (where R was —Cl) and acrylamide were added to a 100 mL three-neck flask at a molar ratio of 1:1.5, then toluene was added to make the yellow 2′ and the acrylamide have a concentration of 0.02 mol/L and 0.03 mol/L, respectively, and a niobium pentoxide catalyst that was 50% of a molar equivalent of the yellow 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, yellow 4.

### Example 9: A Fluorane Chromotropic Dye Structure with Good Light Resistance and a Synthetic Method Therefor

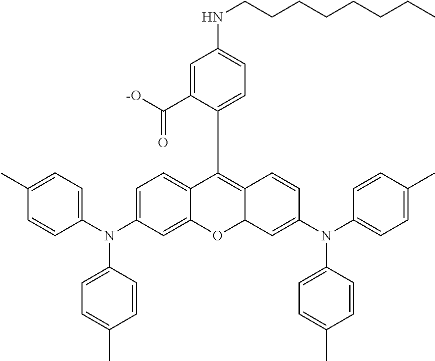

Under the protection of N₂, blue 1′ (where R was —Cl) and octylamine were added to a 100 mL three-neck flask at a molar ratio of 1:1.5, then toluene was added to make the blue 1′ and the octylamine have a concentration of 0.02 mol/L and 0.03 mol/L, respectively, and a niobium pentoxide catalyst that was 50% of a molar equivalent of the blue 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, blue 1.

### Example 10: A Fluorane Chromotropic Dye Structure with Good Light Resistance and a Synthetic Method Therefor

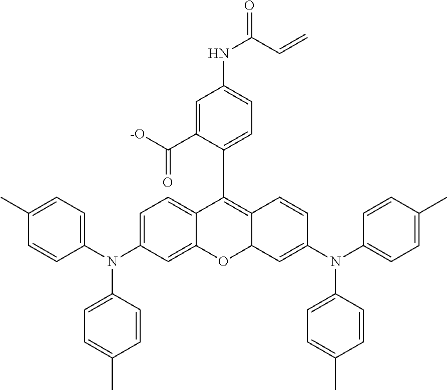

Under the protection of N₂, blue 1′ (where R was —Cl) and acrylamide were added to a 100 mL three-neck flask at a molar ratio of 1:1.5, then toluene was added to make the blue 1′ and the acrylamide have a concentration of 0.02 mol/L and 0.03 mol/L, respectively, and a niobium pentoxide catalyst that was 50% of a molar equivalent of the blue 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, blue 2.

### Example 11: A Fluorane Chromotropic Dye Structure with Good Light Resistance and a Synthetic Method Therefor

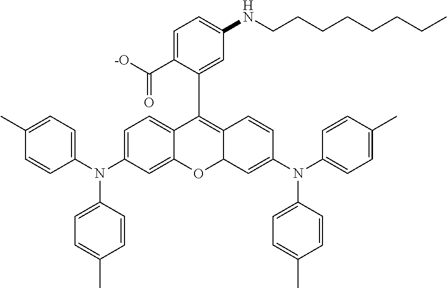

Under the protection of N₂, blue 2′ (where R was —Cl) and octylamine were added to a 100 mL three-neck flask at a molar ratio of 1:1.5, then toluene was added to make the blue 2′ and the octylamine have a concentration of 0.02 mol/L and 0.03 mol/L, respectively, and a niobium pentoxide catalyst that was 50% of a molar equivalent of the blue 2′ was added. Then, ae pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, blue 3.

### Example 12: A Fluorane Chromotropic Dye Structure with Good Light Resistance and a Synthetic Method Therefor

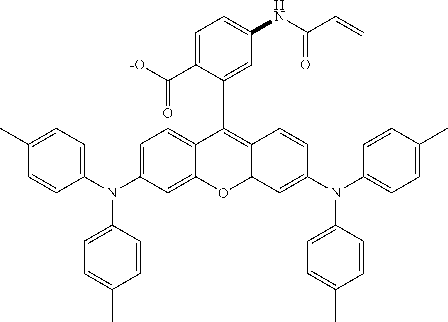

Under the protection of N₂, blue 2′ (where R was —Cl) and acrylamide were added to a 100 mL three-neck flask at a molar ratio of 1:1.5, then toluene was added to make the blue 2′ and the acrylamide have a concentration of 0.02 mol/L and 0.03 mol/L, respectively, and a niobium pentoxide catalyst that was 50% of a molar equivalent of the blue 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, blue 4.

### Example 13: A Fluorane Chromotropic Dye Structure with Good Light Resistance and a Synthetic Method Therefor

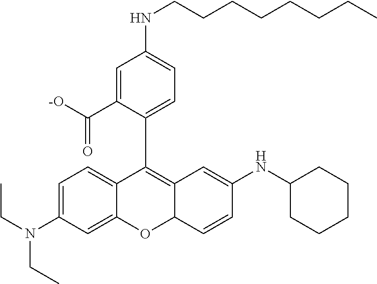

Under the protection of N₂, black 1′ (where R was —Cl) and octylamine were added to a 100 mL three-neck flask at a molar ratio of 1:1.5, then toluene was added to make the black 1′ and the octylamine have a concentration of 0.02 mol/L and 0.03 mol/L, respectively, and a niobium pentoxide catalyst that was 50% of a molar equivalent of the black 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, black 1.

### Example 14: A Fluorane Chromotropic Dye Structure with Good Light Resistance and a Synthetic Method Therefor

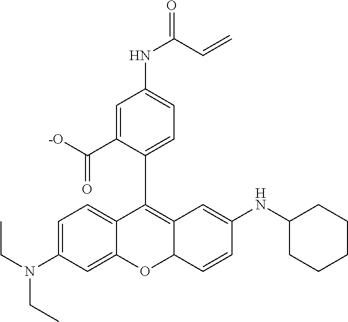

Under the protection of N₂, black 1′ (where R was —Cl) and acrylamide were added to a 100 mL three-neck flask at a molar ratio of 1:1.5, then toluene was added to make the black 1′ and the acrylamide have a concentration of 0.02 mol/L and 0.03 mol/L, respectively, and a niobium pentoxide catalyst that was 50% of a molar equivalent of the black 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, black 2.

### Example 15: A Fluorane Chromotropic Dye Structure with Good Light Resistance and a Synthetic Method Therefor

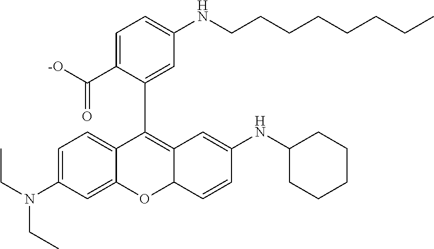

Under the protection of N₂, black 2′ (where R was —Cl) and octylamine were added to a 100 mL three-neck flask at a molar ratio of 1:1.5, then toluene was added to make the black 2′ and the octylamine have a concentration of 0.02 mol/L and 0.03 mol/L, respectively, and a niobium pentoxide catalyst that was 50% of a molar equivalent of the black 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, black 3.

### Example 16: A Fluorane Chromotropic Dye Structure with Good Light Resistance and a Synthetic Method Therefor

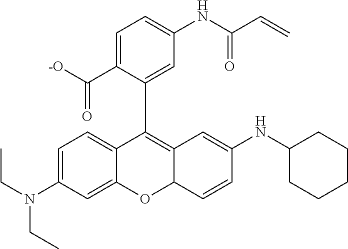

Under the protection of N₂, black 2′ (where R was —Cl) and acrylamide were added to a 100 mL three-neck flask at a molar ratio of 1:1.5, then toluene was added to make the black 2′ and the acrylamide have a concentration of 0.02 mol/L and 0.03 mol/L, respectively, and a niobium pentoxide catalyst that was 50% of a molar equivalent of the black 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, black 4.

### Example 17: A Fluorane Chromotropic Dye Structure with Good Light Resistance and a Synthetic Method Therefor

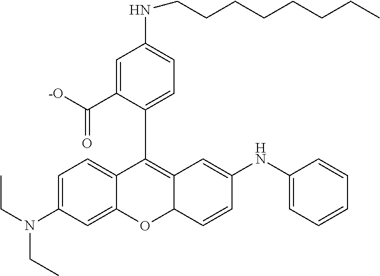

Under the protection of N₂, green 1′ (where R was —Cl) and octylamine were added to a 100 mL three-neck flask at a molar ratio of 1:1.5, then toluene was added to make the green 1′ and the octylamine have a concentration of 0.02 mol/L and 0.03 mol/L, respectively, and a niobium pentoxide catalyst that was 50% of a molar equivalent of the green 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, green 1.

### Example 18: A Fluorane Chromotropic Dye Structure with Good Light Resistance and a Synthetic Method Therefor

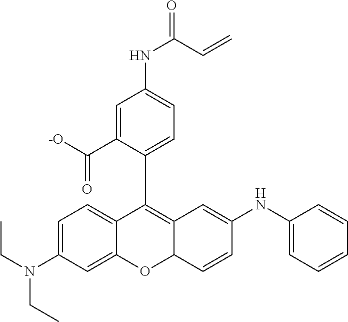

Under the protection of N₂, green 1′ (where R was —Cl) and acrylamide were added to a 100 mL three-neck flask at a molar ratio of 1:1.5, then toluene was added to make the green 1′ and the acrylamide have a concentration of 0.02 mol/L and 0.03 mol/L, respectively, and a niobium pentoxide catalyst that was 50% of a molar equivalent of the green 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, green 2.

### Example 19: A Fluorane Chromotropic Dye Structure with Good Light Resistance and a Synthetic Method Therefor

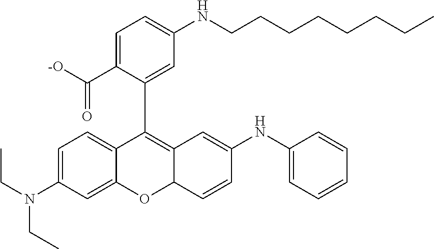

Under the protection of N₂, green 2′ (where R was —Cl) and octylamine were added to a 100 mL three-neck flask at a molar ratio of 1:1.5, then toluene was added to make the green 2′ and the octylamine have a concentration of 0.02 mol/L and 0.03 mol/L, respectively, and a niobium pentoxide catalyst that was 50% of a molar equivalent of the green 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, green 3.

### Example 20: A Fluorane Chromotropic Dye Structure with Good Light Resistance and a Synthetic Method Therefor

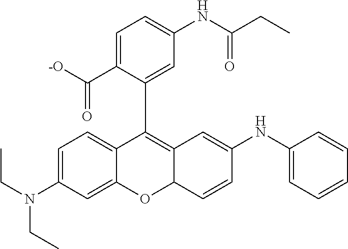

Under the protection of N₂, green 2′ (where R was —Cl) and acrylamide were added to a 100 mL three-neck flask at a molar ratio of 1:1.5, then toluene was added to make the green 2′ and the acrylamide have a concentration of 0.02 mol/L and 0.03 mol/L, respectively, and a niobium pentoxide catalyst that was 50% of a molar equivalent of the green 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, green 4.

### Example 21: A Fluorane Chromotropic Dye Structure with Good Light Resistance and a Synthetic Method Therefor

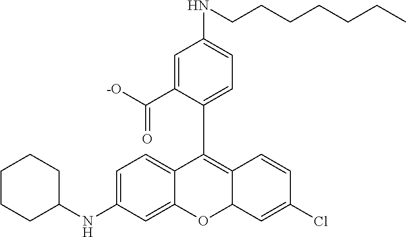

Under the protection of N₂, orange 1′ (where R was —Cl) and octylamine were added to a 100 mL three-neck flask at a molar ratio of 1:1.5, then toluene was added to make the orange 1′ and the octylamine have a concentration of 0.02 mol/L and 0.03 mol/L, respectively, and a niobium pentoxide catalyst that was 50% of a molar equivalent of the orange 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, orange 1.

### Example 22: A Fluorane Chromotropic Dye Structure with Good Light Resistance and a Synthetic Method Therefor

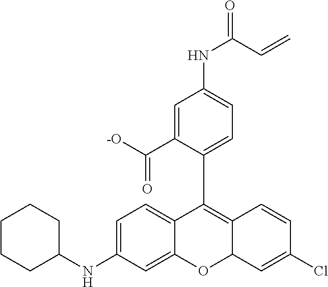

Under the protection of N₂, orange 1′ (where R was —Cl) and acrylamide were added to a 100 mL three-neck flask at a molar ratio of 1:1.5, then toluene was added to make the orange 1′ and the acrylamide have a concentration of 0.02 mol/L and 0.03 mol/L, respectively, and a niobium pentoxide catalyst that was 50% of a molar equivalent of the orange 1′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, orange 2.

### Example 23: A Fluorane Chromotropic Dye Structure with Good Light Resistance and a Synthetic Method Therefor

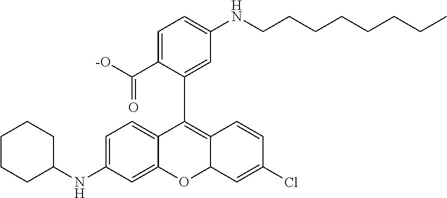

Under the protection of N₂, orange 2′ (where R was —Cl) and octylamine were added to a 100 ml three-neck flask at a molar ratio of 1:1.5, then toluene was added to make the orange 2′ and the octylamine have a concentration of 0.02 mol/L and 0.03 mol/L, respectively, and a niobium pentoxide catalyst that was 50% of a molar equivalent of the orange 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, orange 3.

### Example 24: A Fluorane Chromotropic Dye Structure with Good Light Resistance and a Synthetic Method Therefor

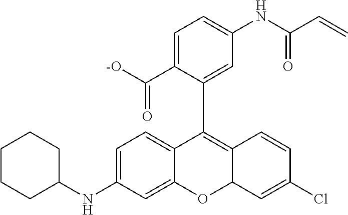

Under the protection of N₂, orange 2′ (where R was —Cl) and acrylamide were added to a 100 mL three-neck flask at a molar ratio of 1:1.5, then toluene was added to make the orange 2′ and the acrylamide have a concentration of 0.02 mol/L and 0.03 mol/L, respectively, and a niobium pentoxide catalyst that was 50% of a molar equivalent of the orange 2′ was added. Then, a pH value was adjusted to 8 with triethylamine, continuous mechanical stirring was performed, and the temperature was raised to 80° C. to carry out a reaction for 8 h. During the reaction, detection was continuously performed by TLC (V_{MeOH}:V_{DCM}=1:10 as a developing agent), and when it was found in an ultraviolet dark box at a waveband of 365 that a raw material dot (R_{f}: 0.35) disappeared and a new orange-yellow fluorescent dot (R_{f}: 0.45) appeared, the reaction was completed. Then, purification was performed by column chromatography with MeOH and DCM (V_{MeOH}:V_{DCM}=1:10) as an eluting solvent to obtain an octylamino substituted fluorane chromotropic dye, orange 4.

### Example 25: A Chromotropic Microcapsule

Provided is a preparation method for a chromotropic microcapsule. The method includes the following steps:
(1) preparation of a prepolymer: taking and placing urea and a formaldehyde solution in a three-neck flask at a molar ratio of 1:1, performing stirring to make the urea dissolved, then dropping triethanolamine to adjust a pH value to 8.5, slowly raising the temperature to 80° C., and carrying out a reaction at a constant temperature for 1 h to obtain a viscous transparent urea-formaldehyde prepolymer;
(2) dispersion of a capsule core: adopting a thermosensitive dye complex as a capsule core having the composition of the fluorane chromotropic dye prepared in Examples 1-24, bisphenol A and a solvent at a mass ratio of 1:4:60, where the solvent was a mixed solution of tetradecanol and hexadecanol at a volume ratio of 4:6; taking and placing the capsule core and water in a flask at a volume ratio of 1:5, performing emulsion dispersion by a high-speed shearing dispersing machine at a speed of 2,000 r/min in a water bath at 60° C. for 1 h, and then performing continued dispersion in a water bath at 30° C. for 1 h to form an O/W type emulsion; and
(3) microencapsulation and post-treatment: mixing the viscous transparent urea-formaldehyde prepolymer with the O/W type emulsion at a volume ratio of 7:1 to obtain a mixed solution, and then adding a mixture of sodium chloride and silica that accounted for 0.5 wt % of a total mass of the mixed solution, where a mass ratio of the sodium chloride to the silica was 1:1; performing full stirring, and then slowly adding acetic acid to adjust the pH value to 3 under the condition of 40° C.; and after a reaction was carried out for 1 h, performing heating to 65° C. to carry out the reaction continuously for 30 min, followed by cooling to room temperature, washing, filtering and drying to obtain a chromotropic microcapsule.

### Example 26: A Preparation Method for a Sunlight-Resistant Chromotropic Fabric

A fabric was dyed with the chromotropic microcapsule prepared from the fluorane chromotropic dye synthesized in Examples 1-24 according to Example 25. Dyeing conditions were that a thickener, an adhesive, 80% of the chromotropic microcapsule and deionized water were blended according to a mass ratio of 1:10:80:9 and then stirred for 30 min to prepare a thermochromic color paste. The fabric was fixed and then subjected to printing treatment by using a screen printing technology, then the printed fabric was dried in an oven at 80° C., and curing was performed at 100° C. for 5 min to obtain a sunlight-resistant chromotropic fabric.

### Comparative Example 1: Synthesis and Purification of Rhodamine B

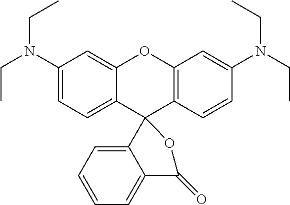 [SMILES: CCN(CC)c1ccc2c(c1)Oc1cc(N(CC)CC)ccc1C21OC(=O)c2ccccc21]

Under the protection of N₂, 2.96 g of phthalic anhydride and 6.61 g of m-hydroxy-N, N-diethylaniline were added, and a niobium pentoxide catalyst that was 50% of a molar equivalent of the phthalic anhydride was added. Then, continuous stirring was performed, and the temperature was raised to 160° C. to carry out a reaction for 3 h. During the reaction, detection was continuously performed by TLC, and when it was found in an ultraviolet dark box at a waveband of 365 that a raw material dot disappeared and a new orange-yellow fluorescent dot appeared, the reaction was completed. Then, a solid was broken, the solid was washed twice with hot saturated salt water, dissolved in methanol and filtered to remove the catalyst, and then an organic phase was concentrated by rotary evaporation. A silica column chromatography powder was added and evenly stirred, drying was performed by rotary evaporation, and separation and purification were performed by column chromatography to obtain Rhodamine B.

### Comparative Example 2: Synthesis and Purification of an Aminofluorane Dye

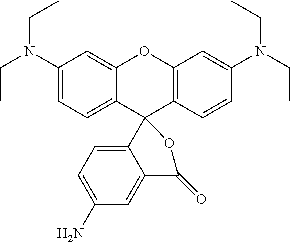 [SMILES: CCN(CC)c1ccc2c(c1)Oc1cc(N(CC)CC)ccc1C21OC(=O)c2cc(N)ccc21]

Under the protection of N₂, 3.26 g of 4-aminophthalic anhydride and 6.61 g of m-hydroxy-N, N-diethylaniline were added, and a niobium pentoxide catalyst that was 50% of a molar equivalent of the 4-aminophthalic anhydride was added. Then, continuous stirring was performed, and the temperature was raised to 160° C. to carry out a reaction for 3 h. During the reaction, detection was continuously performed by TLC, and when it was found in an ultraviolet dark box at a waveband of 365 that a raw material dot disappeared and a new orange-yellow fluorescent dot appeared, the reaction was completed. Then, a solid was broken, the solid was washed twice with hot saturated salt water, dissolved in methanol and filtered to remove the catalyst, and then an organic phase was concentrated by rotary evaporation. A silica column chromatography powder was added and evenly stirred, drying was performed by rotary evaporation, and separation and purification were performed by column chromatography to obtain a 5-aminofluorane dye.

### Comparative Example 3: Synthesis and Purification of a Red 5-Bromofluorane Dye

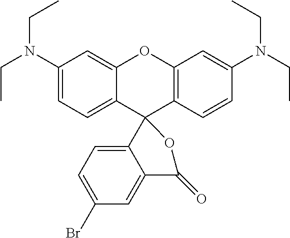 [SMILES: CCN(CC)c1ccc2c(c1)Oc1cc(N(CC)CC)ccc1C21OC(=O)c2cc(Br)ccc21]

Under the protection of N₂, 4.54 g of 4-bromophthalic anhydride and 6.61 g of m-hydroxy-N,N-diethylaniline were added, and a niobium pentoxide catalyst that was 50% of a molar equivalent of the 4-bromophthalic anhydride was added. Then, continuous stirring was performed, and the temperature was raised to 160° C. to carry out a reaction for 3 h. During the reaction, detection was continuously performed by TLC, and when it was found in an ultraviolet dark box at a waveband of 365 that a raw material dot disappeared and a new fluorescent dot appeared, the reaction was completed. Then, a solid was broken, the solid was washed twice with hot saturated salt water, dissolved in methanol and filtered to remove the catalyst, and then an organic phase was concentrated by rotary evaporation. A silica column chromatography powder was added and evenly stirred, drying was performed by rotary evaporation, and separation and purification were performed by column chromatography to obtain a red 5-bromofluorane dye.

### Comparative Example 4: Synthesis and Purification of a Yellow 5-Bromofluorane Dye

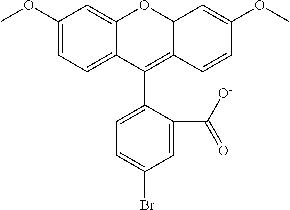

Under the protection of N₂, 4.54 g of 4-bromophthalic anhydride and 2.48 g of m-methoxyphenol were added, and a niobium pentoxide catalyst that was 50% of a mol equivalent of the 4-bromophthalic anhydride was added. Then, continuous stirring was performed, and the temperature was raised to 160° C. to carry out a reaction for 3 h. During the reaction, detection was continuously performed by TLC, and when it was found in an ultraviolet dark box at a waveband of 365 that a raw material dot disappeared and a new fluorescent dot appeared, the reaction was completed. Then, a solid was broken, the solid was washed twice with hot saturated salt water, dissolved in methanol and filtered to remove the catalyst, and then an organic phase was concentrated by rotary evaporation. A silica column chromatography powder was added and evenly stirred, drying was performed by rotary evaporation, and separation and purification were performed by column chromatography to obtain a yellow 5-bromofluorane dye.

### Comparative Example 5: Synthesis and Purification of a Blue 5-Bromofluorane Dye

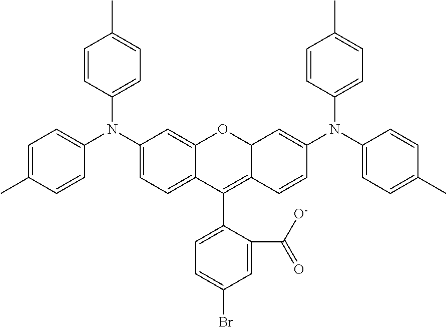

Under the protection of N₂, 4.54 g of 4-bromophthalic anhydride and 11.61 g of m-hydroxy-N,N-di(p-methylphenyl) aniline were added, and a niobium pentoxide catalyst that was 50% of a mol equivalent of the 4-bromophthalic anhydride was added. Then, continuous stirring was performed, and the temperature was raised to 160° C. to carry out a reaction for 3 h. During the reaction, detection was continuously performed by TLC, and when it was found in an ultraviolet dark box at a waveband of 365 that a raw material dot disappeared and a new fluorescent dot appeared, the reaction was completed. Then, a solid was broken, the solid was washed twice with hot saturated salt water, dissolved in methanol and filtered to remove the catalyst, and then an organic phase was concentrated by rotary evaporation. A silica column chromatography powder was added and evenly stirred, drying was performed by rotary evaporation, and separation and purification were performed by column chromatography to obtain a blue 5-bromofluorane dye.

### Comparative Example 6: Synthesis and Purification of a Black 5-Bromofluorane Dye

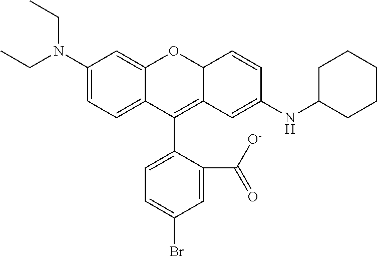 [SMILES: CCN(CC)c1ccc2c(c1)OC1C=CC(NC3CCCCC3)=CC1=C2c1ccc(Br)cc1C(=O)[O-]]

Step (1): In an environmental atmosphere of nitrogen, 0.662 g of m-hydroxy-N,N-diethylaniline and 0.908 g of 4-bromophthalic anhydride (at a molar ratio of 1:1) were taken, 50 mL of a toluene solvent and 0.5 g of a niobium pentoxide catalyst were added, and heating was performed for reflux to carry out a reaction for 4 h. After the reaction was completed, a mixture was cooled to 50° C., and 5 ml of a 35% NaOH aqueous solution was added to the mixture. Then, the mixture was heated to 90° C. and subjected to heat preservation for 6 h. Finally, the mixture was poured into ice, and the resulting mixture was acidified with concentrated hydrochloric acid and then placed at room temperature for 2 h. A suspension was filtered, a solid was recrystallized with ethanol, and the solid was dried to obtain an intermediate 1.

Step (2): 0.392 g of the intermediate 1 and 0.263 g of ODB-1 were weighed according to a molar ratio of 1:1.5 and evenly stirred in 50 ml of a toluene solvent, 0.5 g of a niobium pentoxide catalyst was added, and then a mixture was heated for reflux to carry out a reaction for 5 h and remove water generated in the process. After the reaction was completed, a resulting organic phase was concentrated with 20 ml of a 25% NaOH aqueous solution to recover the solvent, and a residue was recrystallized with ethanol to obtain a black 5-bromofluorane dye.

### Comparative Example 7: Synthesis and Purification of a Green 5-Bromofluorane Dye

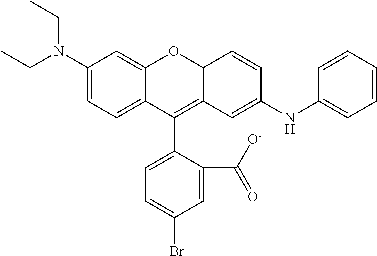 [SMILES: CCN(CC)c1ccc2c(c1)OC1C=CC(Nc3ccccc3)=CC1=C2c1ccc(Br)cc1C(=O)[O-]]

Step (1): In an environmental atmosphere of nitrogen, 0.662 g of m-hydroxy-N,N-diethylaniline and 0.908 g of 4-bromophthalic anhydride (at a molar ratio of 1:1) were taken, then 50 ml of a toluene solvent and 0.5 g of a niobium pentoxide catalyst were added, and heating was performed for reflux to carry out a reaction for 4 h. After the reaction was completed, a mixture was cooled to 50° C., and 5 mL of a 35% NaOH aqueous solution was added to the mixture. Then, the mixture was heated to 90° C. and subjected to heat preservation for 6 h. Finally, the mixture was poured into ice, and the resulting mixture was acidified with concentrated hydrochloric acid and then placed at room temperature for 2 h. A suspension was filtered, a solid was recrystallized with ethanol, and the solid was dried to obtain an intermediate 2.

Step (2): 0.392 g of the intermediate 2 and 0.254 g of ODB-2 were weighed according to a molar ratio of 1:1.5 and evenly stirred in 50 mL of a toluene solvent, 0.5 g of a niobium pentoxide catalyst was added, and then a mixture was heated for reflux to carry out a reaction for 5 h and remove water generated in the process. After the reaction was completed, a resulting organic phase was concentrated with 20 mL of a 25% NaOH aqueous solution to recover the solvent, and a residue was recrystallized with ethanol to obtain a green 5-bromofluorane dye.

### Comparative Example 8: Synthesis and Purification of an Orange 5-Bromofluorane Dye

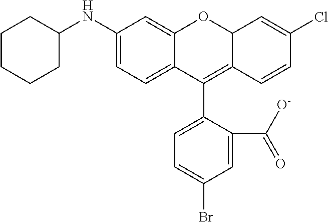 [SMILES: O=C([O-])c1cc(Br)ccc1C1=C2C=CC(Cl)=CC2Oc2cc(NC3CCCCC3)ccc21]

Step (1): In an environmental atmosphere of nitrogen, 0.384 g of PSD-1 and 0.454 g of 4-bromophthalic anhydride (at a molar ratio of 1:1) were taken, 50 ml of a toluene solvent and 0.5 g of a niobium pentoxide catalyst were added, and heating was performed for reflux to carry out a reaction for 4 h. After the reaction was completed, a mixture was cooled to 50° C., and an appropriate amount of a 35% NaOH aqueous solution was added to the mixture. Then, the mixture was heated to 90° C. and subjected to heat preservation for 6 h. Finally, the mixture was poured into ice, and the resulting mixture was acidified with concentrated hydrochloric acid and then placed at room temperature for 2 h. A suspension was filtered, a solid was recrystallized with ethanol, and the solid was dried to obtain an intermediate 3.

Step (2): 0.419 g of the intermediate 3 and 0.167 g of ODB-3 were weighed according to a molar ratio of 1:1.5 and evenly stirred in 50 ml of a toluene solvent, 0.5 g of a niobium pentoxide catalyst was added, and then a mixture was heated for reflux to carry out a reaction for 5 h and remove water generated in the process. After the reaction was completed, a resulting organic phase was concentrated with an appropriate amount of a 25% NaOH aqueous solution to recover the solvent, and a residue was recrystallized with ethanol to obtain an orange 5-bromofluorane dye.

### Comparative Example 9:6-Amido Substituted Chromotropic Fluorane Dye

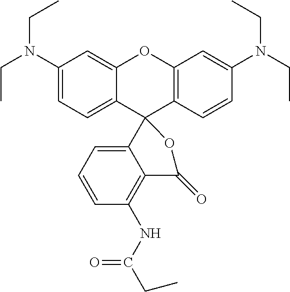 [SMILES: CCC(=O)Nc1cccc2c1C(=O)OC21c2ccc(N(CC)CC)cc2Oc2cc(N(CC)CC)ccc21]

Step (1): 50 mmol of 3-(diethylamino) phenol and 25 mmol of 3-nitrophthalic anhydride were added to 50 mL of chlorobenzene for complete dissolution, and then 25 mmol of trifluoromethanesulfonic acid was added. Then, preheating was performed in an oil bath pot at 135° C., reflux was performed to carry out a reaction for 2 days in an environmental atmosphere of nitrogen, cooling was performed to room temperature after the reaction was completed, and a solvent was removed by a rotary evaporation method. Column chromatography was performed with dichloromethane/methanol as an eluting agent to obtain a purplish red powder, namely a product ph-NO₂.

Step (2): 1 mmol of the ph-NO₂, 20 mg of Pd/C and 4 mL of ethyl acetate were placed in a three-neck flask. 1.5 mmol of H₃PO₂ and 4.5 mmol of NaH₂PO₂·H₂O were evenly dissolved in 4 mL of H₂O and then poured into the three-neck flask. Then, the three-neck flask was placed in an oil bath pot to carry out a reaction at 85° C. for 5 h, cooling was performed to room temperature, and then extraction was performed with dichloromethane. An extracted product was dried in anhydrous sodium sulfate and then subjected to column chromatography with petroleum ether/ethyl acetate/triethylamine as an eluting agent to obtain a pink powder, namely a product ph-NH₂.

Step (3): 2 mmol of the ph-NH₂ and 3 mmol of triethylamine were dissolved in 10 mL of DCM, and 12 mmol of propionyl chloride was slowly dropped in an ice water bath to carry out a reaction at room temperature for 2 h. A solvent was removed by rotary evaporation, and column chromatography was performed with petroleum ether/ethyl acetate/triethylamine as an eluting agent to obtain a light pink powder, namely a final product, which was a red target fluorane dye.

### Comparative Example 10:6-Amido Substituted Chromotropic Fluorane Dye

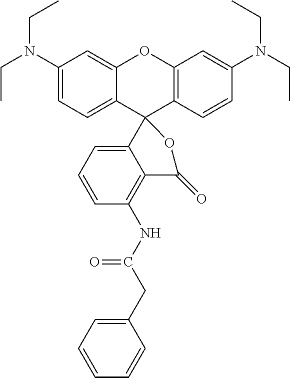 [SMILES: CCN(CC)c1ccc2c(c1)Oc1cc(N(CC)CC)ccc1C21OC(=O)c2c(NC(=O)Cc3ccccc3)cccc21]

Step (1): 50 mmol of 3-(diethylamino) phenol and 25 mmol of 3-nitrophthalic anhydride were added to a three-neck flask filled with 50 ml of chlorobenzene for complete dissolution, and then 25 mmol of trifluoromethanesulfonic acid was added. Then, the three-neck flask was placed in an oil bath pot for preheating at 135° C., reflux was performed to carry out a reaction for 2 days in an environmental atmosphere of nitrogen, cooling was performed to room temperature after the reaction was completed, and a solvent was removed by a rotary evaporation method. Column chromatography was performed with dichloromethane/methanol as an eluting agent to obtain a purplish red powder, namely a product ph-NO₂.

Step (2): 1 mmol of the ph-NO₂, 20 mg of Pd/C and 4 mL of ethyl acetate (EtOAc) were placed in a three-neck flask. 1.5 mmol of H₃PO₂ and 4.5 mmol of NaH₂PO₂·H₂O were evenly dissolved in 4 mL of H₂O and then poured into the three-neck flask Then, the three-neck flask was placed in an oil bath pot to carry out a reaction at 85° C. for 5 h, cooling was performed to room temperature, and then extraction was performed with dichloromethane. An extracted product was dried in anhydrous sodium sulfate and then subjected to column chromatography with petroleum ether/ethyl acetate/triethylamine as an eluting agent to obtain a pink powder, namely a product ph-NH₂.

Step (3): 2 mmol of the ph-NH₂ and 3 mmol of triethylamine were dissolved in 10 mL of DCM, and 12 mmol of phenylacetyl chloride was slowly dropped in an ice water bath to carry out a reaction at room temperature for 2 h. A solvent was removed by rotary evaporation, and column chromatography was performed with petroleum ether/ethyl acetate/triethylamine as an eluting agent to obtain a light pink powder, namely a final product, which was a red target fluorane dye.

### Comparative Example 11:6-Phenylamido Substituted Chromotropic Fluorane Dye

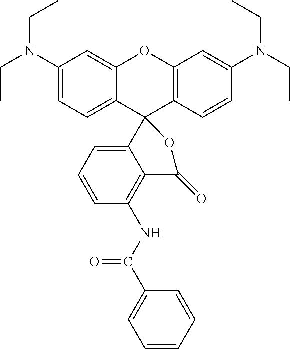 [SMILES: CCN(CC)c1ccc2c(c1)Oc1cc(N(CC)CC)ccc1C21OC(=O)c2c(NC(=O)c3ccccc3)cccc21]

Step (1): 50 mmol of 3-(diethylamino) phenol and 25 mmol of 3-nitrophthalic anhydride were added to 50 mL of chlorobenzene for complete dissolution, and then 25 mmol of trifluoromethanesulfonic acid was added. Then, the compounds were placed in an oil bath pot for preheating at 135° C., reflux was performed to carry out a reaction for 2 days in an environmental atmosphere of nitrogen, cooling was performed to room temperature after the reaction was completed, and a solvent was removed by a rotary evaporation method. Column chromatography was performed with dichloromethane/methanol as an eluting agent to obtain a purplish red powder, namely a product ph-NO₂.

Step (2): 1 mmol of the ph-NO₂, 20 mg of Pd/C and 4 mL of ethyl acetate were placed in a three-neck flask. 1.5 mmol of H₃PO₂ and 4.5 mmol of NaH₂PO₂·H₂O were evenly dissolved in 4 mL of H₂O and then poured into the three-neck flask. Then, the three-neck flask was placed in an oil bath pot to carry out a reaction at 85° C. for 5 h, cooling was performed to room temperature, and then extraction was performed with dichloromethane. An extracted product was dried in anhydrous sodium sulfate and then subjected to column chromatography with petroleum ether/ethyl acetate/triethylamine as an eluting agent to obtain a pink powder, namely a product ph-NH₂.

Step (3): 2 mmol of the ph-NH₂ and 3 mmol of triethylamine were dissolved in 10 mL of DCM, and 12 mmol of benzoyl chloride was slowly dropped in an ice water bath to carry out a reaction at room temperature for 2 h. A solvent was removed by rotary evaporation, and column chromatography was performed with petroleum ether/ethyl acetate/triethylamine as an eluting agent to obtain a light pink powder, namely a final product, which was a red target fluorane dye.

### Comparative Example 12: A Bistable Electrochromic Fluorane Dye

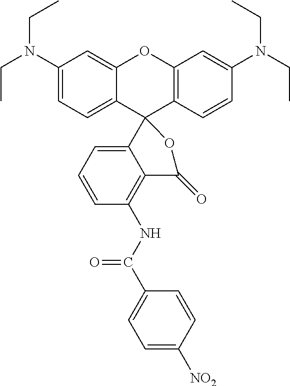 [SMILES: CCN(CC)c1ccc2c(c1)Oc1cc(N(CC)CC)ccc1C21OC(=O)c2c(NC(=O)c3ccc([N+](=O)[O-])cc3)cccc21]

Specific synthesis and purification refer to the patent “Bistable Electrochromic Fluorane Dye and Preparation Method for Device thereof”.

Step (1): 50 mmol of 3-(diethylamino) phenol and 25 mmol of 3-nitrophthalic anhydride were added to 50 ml of chlorobenzene for complete dissolution, and then 25 mmol of trifluoromethanesulfonic acid was added. Then, the compounds were placed in an oil bath pot for preheating at 135° C., reflux was performed to carry out a reaction for 2 days in an environmental atmosphere of nitrogen, cooling was performed to room temperature after the reaction was completed, and a solvent was removed by a rotary evaporation method. Column chromatography was performed with dichloromethane/methanol as an eluting agent to obtain a purplish red powder, namely a product ph-NO₂.

Step (2): 1 mmol of the ph-NO₂, 20 mg of Pd/C and 4 mL of ethyl acetate were placed in a three-neck flask. 1.5 mmol of H₃PO₂ and 4.5 mmol of NaH₂PO₂·H₂O were evenly dissolved in 4 mL of H₂O and then poured into the three-neck flask. Then, the three-neck flask was placed in an oil bath pot to carry out a reaction at 85° C. for 5 h, cooling was performed to room temperature, and then extraction was performed with dichloromethane. An extracted product was dried in anhydrous sodium sulfate and then subjected to column chromatography with petroleum ether/ethyl acetate/triethylamine as an eluting agent to obtain a pink powder, namely a product ph-NH₂.

Step (3): 2 mmol of the ph-NH₂ and 3 mmol of triethylamine were dissolved in 10 mL of DCM, and 12 mmol of p-nitrobenzoyl chloride was slowly dropped in an ice water bath to carry out a reaction at room temperature for 2 h. A solvent was removed by rotary evaporation, and column chromatography was performed with petroleum ether/ethyl acetate/triethylamine as an eluting agent to obtain a light pink powder, namely a final product, which was a red target fluorane dye.

TABLE 1
Sunlight resistance test of fluorane dyes

| | K/S before | 1K/S after | Color fading rate |
|---|---|---|---|
| | sunlight | sunlight | after sunlight |
| | exposure | exposure for 20 h | exposure for 20 h |
| Example 1 | 4.37 | 4.32 | 1.1% |
| Example 2 | 4.33 | 4.25 | 1.8% |
| Example 3 | 4.31 | 4.23 | 1.9% |
| Example 4 | 4.09 | 3.97 | 2.9% |
| Example 5 | 4.17 | 3.99 | 4.3% |
| Example 6 | 4.15 | 4.09 | 1.4% |
| Example 7 | 4.05 | 3.99 | 1.5% |
| Example 8 | 4.09 | 3.94 | 3.7% |
| Example 9 | 4.31 | 4.24 | 1.6% |
| Example 10 | 4.47 | 4.33 | 3.1% |
| Example 11 | 4.15 | 4.08 | 1.7% |
| Example 12 | 4.30 | 4.21 | 2.1% |
| Example 13 | 4.15 | 4.08 | 1.7% |
| Example 14 | 4.54 | 4.45 | 2.0% |
| Example 15 | 4.32 | 4.19 | 3.00% |
| Example 16 | 4.4 | 4.21 | 4.30% |
| Example 17 | 4.38 | 4.31 | 1.60% |
| Example 18 | 4.28 | 4.21 | 1.60% |
| Example 19 | 4.32 | 4.16 | 3.70% |
| Example 20 | 4.43 | 4.37 | 1.40% |
| Example 21 | 4.59 | 4.46 | 2.80% |
| Example 22 | 4.27 | 4.21 | 1.40% |
| Example 23 | 4.42 | 4.34 | 1.80% |
| Example 24 | 4.27 | 4.21 | 1.40% |
| Comparative | 4.34 | 2.96 | 31.8% |
| Example 1 | | | |
| Comparative | 4.36 | 2.51 | 42.4% |
| Example 2 | | | |
| Comparative | 4.27 | 3.45 | 19.2% |
| Example 3 | | | |
| Comparative | 4.39 | 3.51 | 20.0% |
| Example 4 | | | |
| Comparative | 4.56 | 3.60 | 21.0% |
| Example 5 | | | |
| Comparative | 4.61 | 3.59 | 22.1% |
| Example 6 | | | |
| Comparative | 4.58 | 3.62 | 21.0% |
| Example 7 | | | |
| Comparative | 4.31 | 3.52 | 18.3% |
| Example 8 | | | |
| Comparative | 4.23 | 3.36 | 21.0% |
| Example 9 | | | |
| Comparative | 4.46 | 3.29 | 26.2% |
| Example 10 | | | |
| Comparative | 4.67 | 3.27 | 30.0% |
| Example 11 | | | |
| Comparative | 4.78 | 3.34 | 30.1% |
| Example 12 | | | |

Results show that when the R₂ and the R₃ in the structure of the fluorane chromotropic dye are independently selected from hydrogen, ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, and alkyl containing a polar group, such as OH (hydroxyl), COOH (carboxyl), NH (amino), or CO—NH₂ (amido); and the R₂ and the R₃ are not hydrogen simultaneously, and sunlight resistance of the dye can be correspondingly improved.

Although the present disclosure has been disclosed above as preferred examples, the examples are not intended to limit the present disclosure, and various changes and modifications can be made by any person familiar with the art without departing from the spirit and scope of the present disclosure. Therefore, the scope of protection of the present disclosure shall be defined by the claims.

What is claimed is:

1. A fluorane chromotropic dye with good light resistance, wherein the fluorane chromotropic dye has a general structural formula shown in a formula I below: 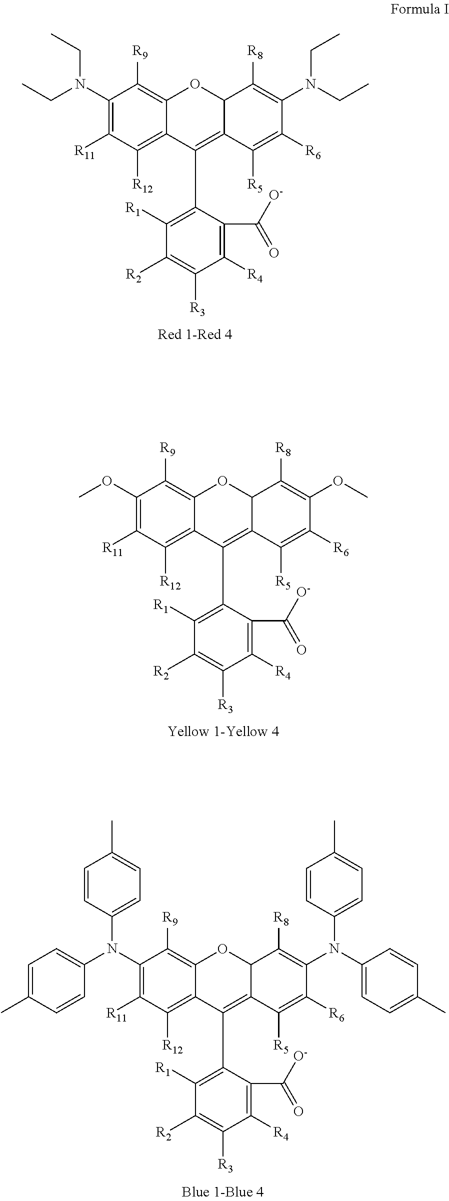 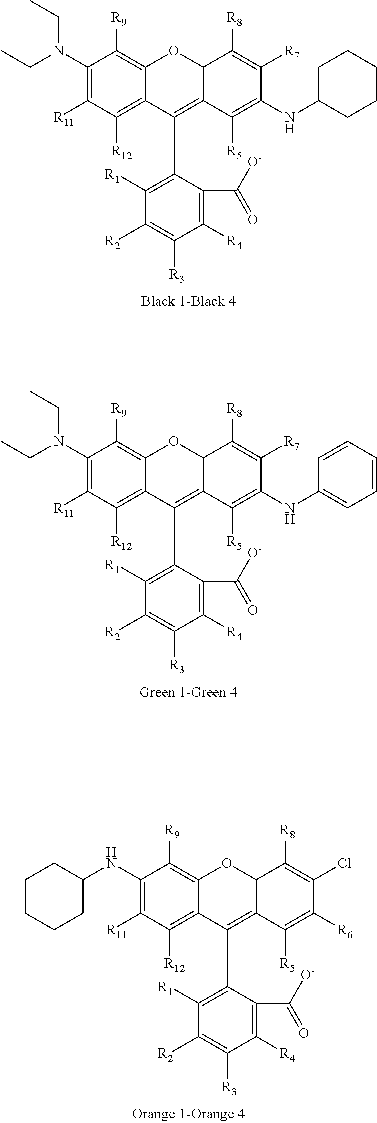
wherein:
R₅, R₆, R₇, R₈, R₉, R₁₁ and R₁₂ are each independently selected from hydrogen, alkyl, substituted alkyl, alkenyl, substituted alkenyl, heterocyclic alkyl, substituted heterocyclic alkyl, alkoxyl, substituted alkoxyl, substituted carbonyl, acylamino, and halogen;
R₁ and R₄ are H; and
R₂ and R₃ are independently selected from hydrogen or alkylamino with different carbon atom numbers, wherein alkyl in the alkylamino is a straight-chain alkane with a carbon atom number of C₂-C₁₂, a cycloalkane, or an alkane containing a polar group; and the R₂ and the R₃ are not hydrogen simultaneously.

2. The fluorane chromotropic dye according to claim 1, wherein the R₂ and the R₃ are independently selected from hydrogen or alkylamino, wherein alkyl in the alkylamino comprises ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, or an alkane containing a polar group; and the R₂ and the R₃ are not hydrogen simultaneously.

3. The fluorane chromotropic dye according to claim 1, wherein the R₂ and the R₃ are independently selected from hydrogen or alkylamino, wherein alkyl in the alkylamino comprises ethyl, propyl and isopropyl, butyl and isomers thereof, pentyl and isomers thereof, hexyl and isomers thereof, heptyl and isomers thereof, octyl and isomers thereof, nonyl and isomers thereof, decyl and isomers thereof, or alkyl containing any one of hydroxyl or carboxyl; and the R₂ and the R₃ are not hydrogen simultaneously.

4. A method for synthesis of the fluorane chromotropic dye according to claim 1, comprising the following steps:
mixing a fluorane chromotropic dye structural intermediate with alkylamine, then adding an organic solvent and a catalyst, performing stirring, adjusting a pH value to 8-9, then carrying out a heating reaction, and after the reaction is completed, performing purification to obtain the fluorane chromotropic dye.

5. The method according to claim 4, wherein the synthetic method is carried out in a protective gas environment.

6. The method according to claim 4, wherein the fluorane chromotropic dye structural intermediate has a general structural formula shown in a formula II below: 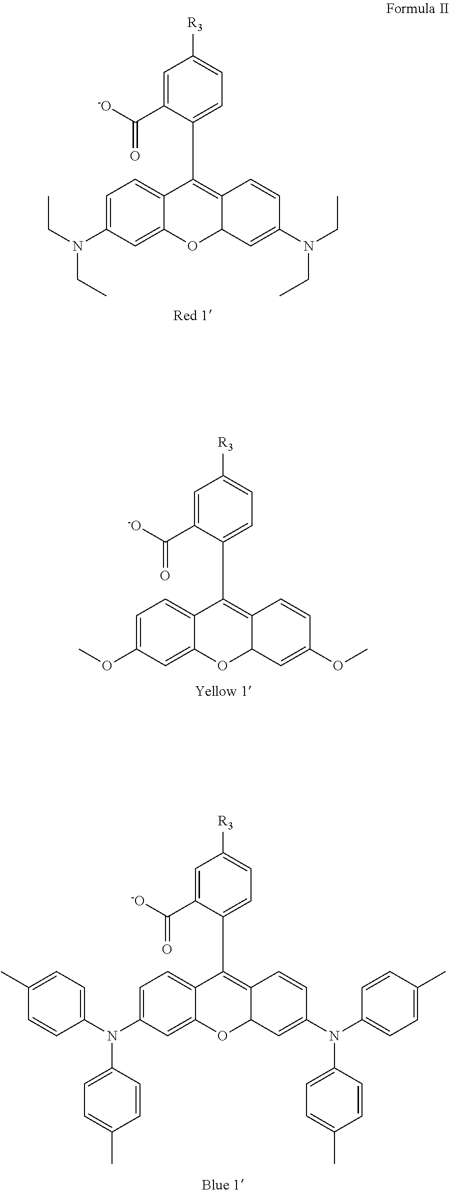 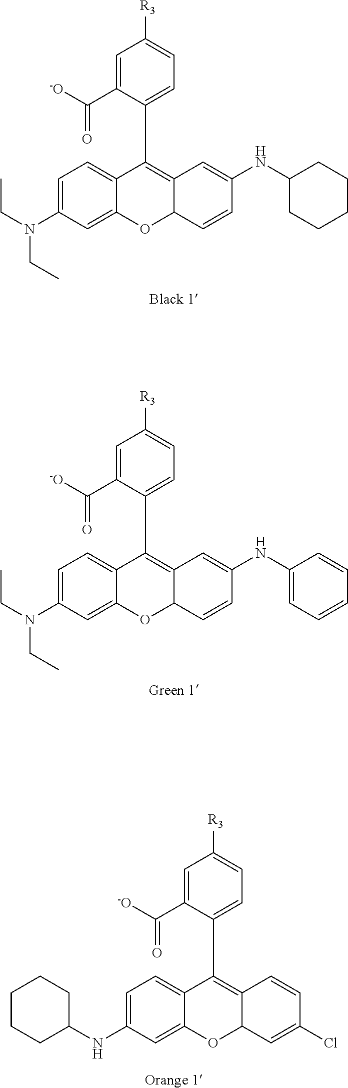
wherein: R₃ is halogen.

7. The method according to claim 4, wherein the fluorane chromotropic dye structural intermediate has a general structural formula shown in a formula III below: 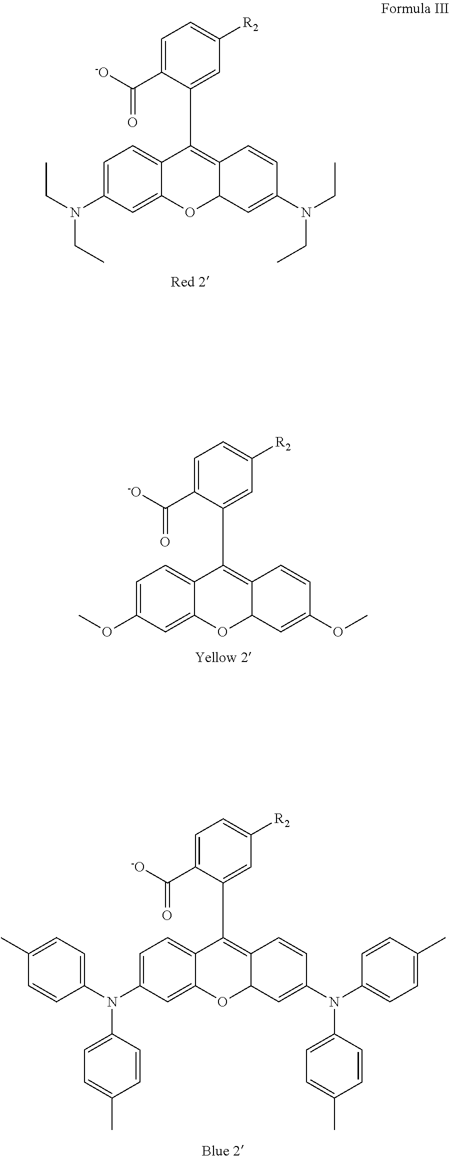 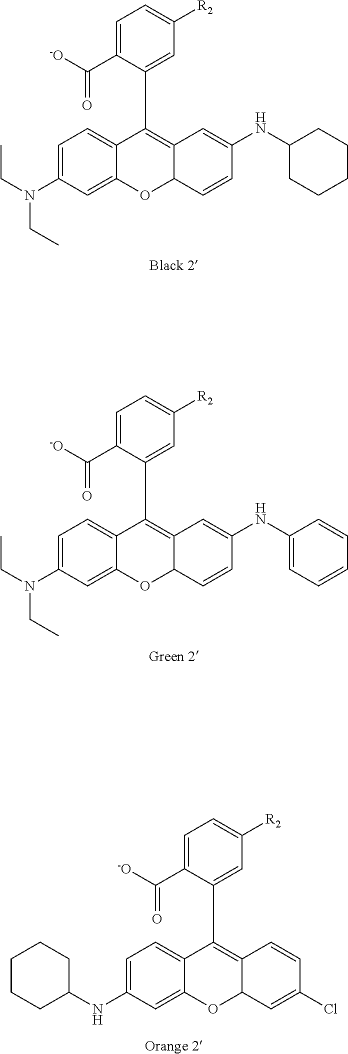
wherein: R₂ is halogen.

8. The method according to claim 4, wherein alkyl in the alkylamine comprises a C₂-C₁₂ straight-chain alkane, a cycloalkane, or an alkyl hydrocarbon group containing a polar group.

9. The method according to claim 4, wherein the organic solvent is toluene; and the catalyst is niobium pentoxide.

10. The method according to claim 4, wherein a molar ratio of the fluorane chromotropic dye structural intermediate to the alkylamine is 1:(1-2); a ratio of a molar amount of the fluorane chromotropic dye structural intermediate to a volume of the organic solvent is 1 mol:(0.05-0.1) mL; and a molar ratio of the fluorane chromotropic dye structural intermediate to the catalyst is 1:(0.5-0.7).

11. The method according to claim 4, wherein the heating reaction is carried out at 75-85° C. for 6-9 hours.

12. A sunlight-resistant chromotropic microcapsule, comprising the fluorane chromotropic dye according to claim 1 as a core material and a high-molecular polymer, an inorganic particle or an inorganic particle doped polymer as a wall material; and a preparation method for the chromotropic microcapsule comprises the following steps:
(1) preparation of a prepolymer: taking urea and a formaldehyde solution for mixing, then adding triethanolamine to adjust the pH value to 8-9, and carrying out a heating reaction to obtain a viscous transparent urea-formaldehyde prepolymer;
(2) dispersion of a capsule core: mixing the fluorane chromotropic dye, bisphenol A and a solvent to prepare the capsule core, then mixing the capsule core with water, performing shearing dispersion emulsification, and performing continued dispersion after cooling to form an O/W type emulsion; and
(3) microencapsulation and post-treatment: mixing the viscous transparent urea-formaldehyde prepolymer with the O/W type emulsion to obtain a mixed solution, then adding sodium chloride and silica for full stirring, then adding acetic acid to adjust the pH value to 3-4, followed by carrying out a heating reaction, and after the reaction is completed, performing cooling, washing, filtering and drying to obtain the chromotropic microcapsule.

13. The sunlight-resistant chromotropic microcapsule according to claim 12, wherein in step (2), the solvent is a mixed solution of tetradecanol and hexadecanol that are mixed at a mass ratio of (4-5):(5-6); a mass ratio of the fluorane chromotropic dye to the bisphenol A is 1:(2-8); a mass ratio of the fluorane chromotropic dye to the solvent is 1:(50-70); and a volume ratio of the capsule core to the water is 1:(4-7).

14. The sunlight-resistant chromotropic microcapsule according to claim 12, wherein in step (3), a volume ratio of the viscous transparent urea-formaldehyde prepolymer to the O/W type emulsion is (5-10): 1; the sodium chloride accounts for 0.2-0.25 wt % of a total mass of the mixed solution; and the silica accounts for 0.2-0.25 wt % of the total mass of the mixed solution.
